(19) United States
(12) Patent Application Publication
(10) Pub. No.: US 2016/0129216 A1
(43) Pub. Date: May 12, 2016

(54) Circulatory Water Flow System

(71) Applicants: Louis O. Leazenbee, Lawrenceville, GA (US); William B. Brimer, Suwanee, GA (US)

(72) Inventors: Louis O. Leazenbee, Lawrenceville, GA (US); William B. Brimer, Suwanee, GA (US)

(21) Appl. No.: 14/536,533

(22) Filed: Nov. 7, 2014

(51) Int. Cl. A61M 21/02; A47K 1/04

(52) U.S. Cl. CPC A61M 21/02; A47K 1/04; A61M 2021/0027

(57) ABSTRACT

A circulatory water flow sensory system having a water nozzle attached to a basin. A pump sends water from the water reservoir through the nozzle and into the basin. The water then drains from the basin into the water reservoir. The basin is shaped in such a way to allow easy and safe access to the flowing water for children, especially those with developmental disorders such as autism.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND

Autism and other pervasive developmental disorders are complex disabilities which affect a person's ability to communicate and interact with the world around them. The CDC estimated that 1.5% of children under the age of 3 were diagnosed with autism in 2014. Many of these children have difficulty functioning in a normal manner which leads to an increase in anxiety levels. This sensory water system was designed to provide a safe environment for any child, including those with developmental disorders, to have the benefit of various sensory experiences. Soothing sounds and flowing water, among other sensory engagement, tend to relax and reduce anxiety in autistic children. For the foregoing reasons, there is a need for a sensory water system which is safe and easy to use for children with autism.

### SUMMARY

The present invention is directed to an apparatus that satisfies the need for a safe and easy to use water system to allow children with autism to engage in various sensory activities, including running water and soothing sounds. The apparatus comprises a base which houses a removable water reservoir and a pump, and a basin on which a nozzle and a control mechanism are attached. The pump pulls water from the water reservoir, pushes it through the nozzle, and into the basin. Once in the basin, the water drains into the water reservoir and the cycle begins again. The apparatus can include a locking mechanism to lock the removable water reservoir into the base. The apparatus can also include a basin with a drain hole positioned so the water drains directly into the removable water reservoir without need of a hose.

### BRIEF DESCRIPTION OF THE DRAWINGS

These and other features, aspects, and advantages of the present invention will become better understood with regard to the following description, appended claims, and accompanying drawings where:

FIG. 1 is an exploded perspective view of an apparatus embodying features of the present invention; and

FIG. 2 is a front view of the apparatus of FIG. 1; and

FIG. 3 is a side view of the apparatus of FIG. 1; and

FIG. 4 is a perspective view with a “cut-away” showing internal elements of the apparatus of FIG. 1; and

FIG. 5 is a top view of the apparatus of FIG. 1.

### DESCRIPTION

In the Summary above and in the Description, and the claims below, and in the accompanying drawings, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally. The term “comprises” and grammatical equivalents thereof are used herein to mean that other components are optionally present. For example, an article “comprising” components A, B, and C can consist of components A, B, and C, or can contain not only components A, B, and C but also one or more other components.

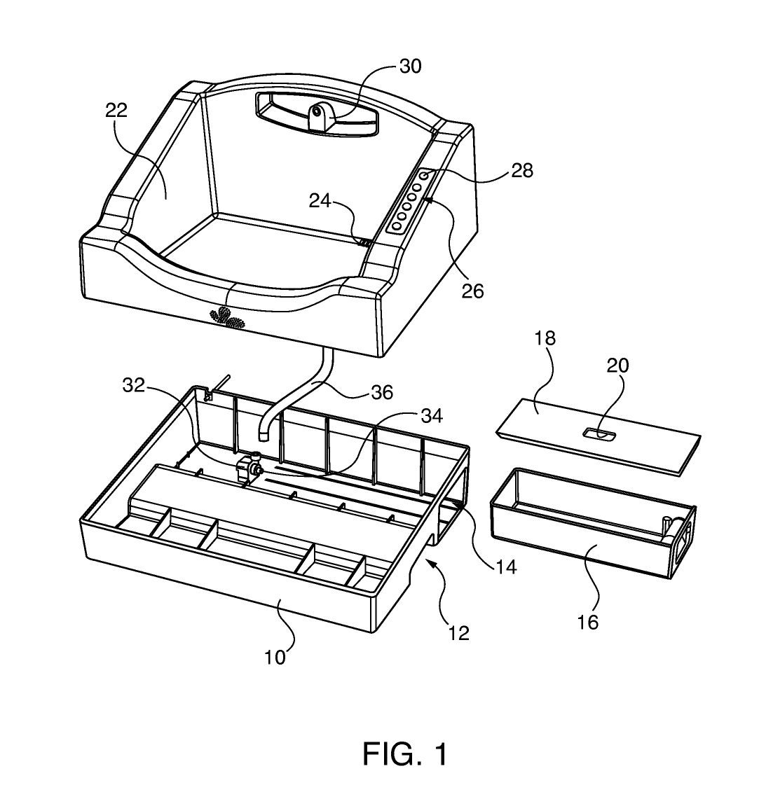

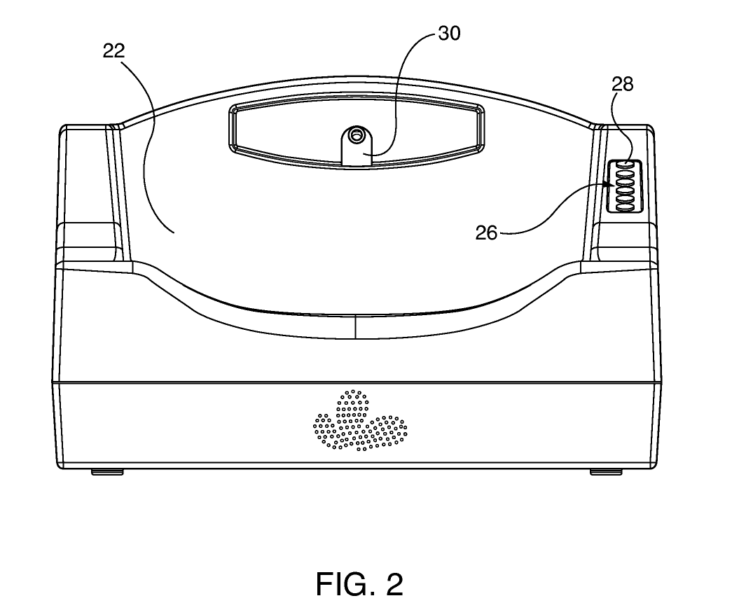

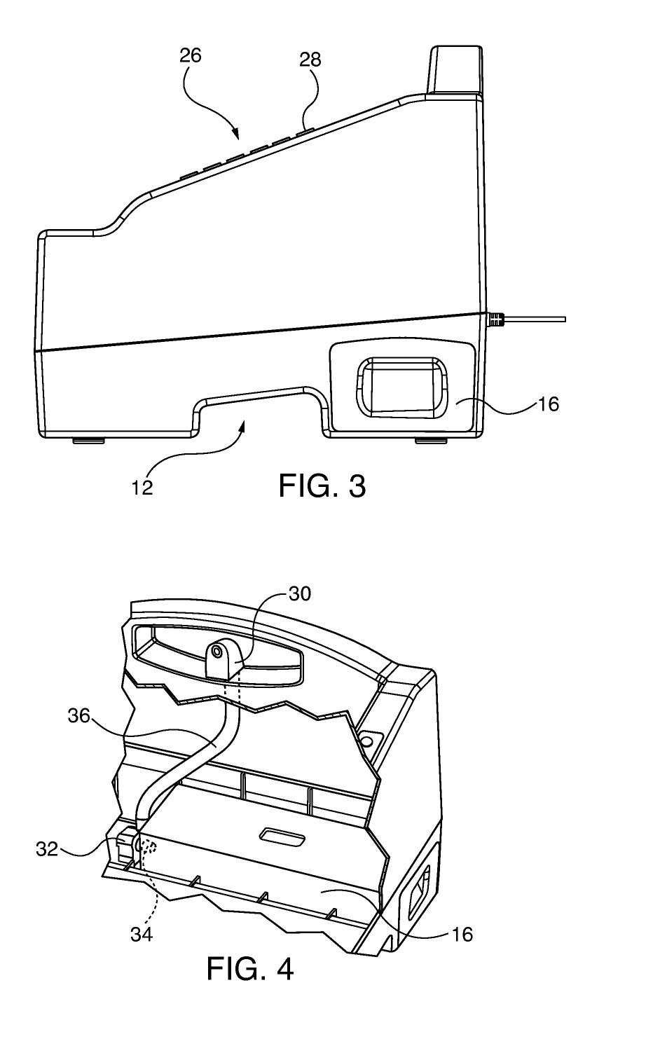

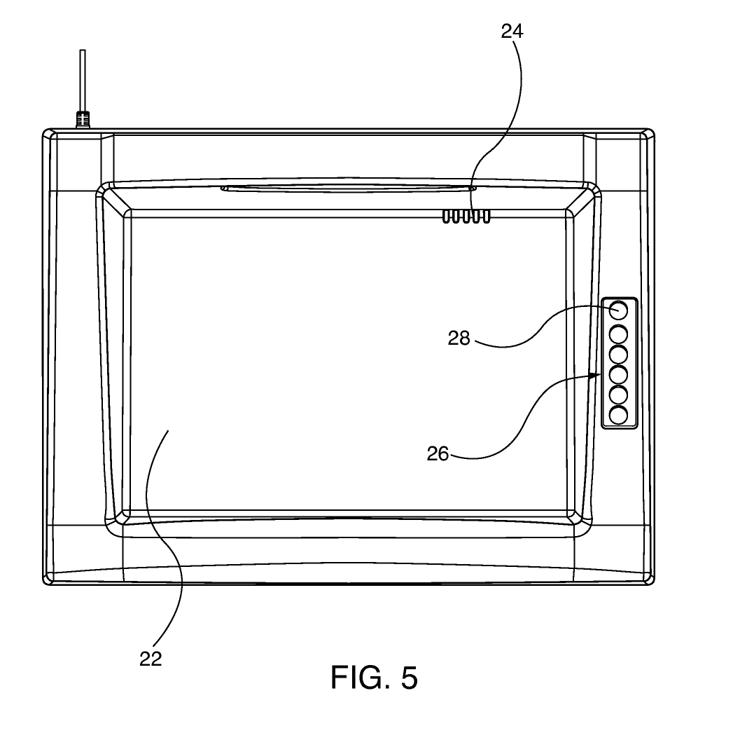

As shown in FIG. 1 a circulatory flow water sensory system comprises a base 10, a water reservoir 16, a basin 22, a control mechanism 26, a nozzle 30, and a pump 32. The base 10 is generally cuboid shaped and comprises an opening 14 on the front, back, or a side to allow insertion of the removable water reservoir 16, a mounting location for the pump 32, and a mounting location for the basin 22. Optionally, the base 10 can also comprise a locking mechanism to lock the removable water reservoir 16 inside the base 10. The removable water reservoir 16 is generally cuboid shaped and comprises an opening 20 in the lid 18 to allow water to pour in and an opening on one side to permit connection to the pump 32 and a removable lid 18. Optionally, the lid 18 of the removable water reservoir 16 can be hinged. The basin 22 is generally shaped like a common household sink but with the front facing wall significantly shorter than the back wall to allow easy access to the water in the basin 22. The basin 22 comprises a mounting location for the nozzle 30, a mounting location for the control mechanism 26, and drain holes 24. The control mechanism 26 is affixed to an outer surface of the basin 22 and is electrically connected to the pump 32. Optionally, the control mechanism 26 can be electrically connected to any number of additional sensory engagement elements. The nozzle 30 is affixed on the basin 22 and connected to the pump 32. The pump 32 is affixed inside the base 10 and connected to the removable water reservoir 16 and nozzle 30. The base 10 is a rectangular block with the longest dimension oriented along the horizontal axis facing the user. The bottom has a convex arch 12 running the entire length horizontally from one side to the other. The base 10 is made from injection molded plastic, but could also be made out of fiberglass or machined aluminum. The purpose is to house the internal wiring for the entire system, as well as house the pump 32 and the removable water reservoir 16. In order to insert and remove the removable water reservoir 16, the base 10 must have an opening 14 located on either the front, back, or a side wall. In an alternative embodiment, the removable water reservoir 16 locking mechanism is built into the base 10 near the water reservoir opening 14.

The removable water reservoir 16 is a hollow rectangular box with a lid 18 on top. The removable water reservoir 16 is made from injection molded plastic, but could also be made out of fiberglass or machined aluminum. The purpose is to store water and to catch water which drains from the basin 22. The lid 18 of the removable water reservoir 16 has an opening 20 which will allow water to drain from the basin 22 into the removable water reservoir 16. The lid opening 20 lines up directly underneath the basin drain hole 24 so that only gravity is needed to drain the water from the basin 22 into the removable water reservoir 16, but draining could also be accomplished using a tube connected from the drain hole to the removable water reservoir 16. The removable water reservoir 16 is removable to allow for quick changing of the water. The lid 18 is also removable to allow quick dumping and refilling of the water once the removable water reservoir 16 is removed, but could also be hinged. The basin 22 is generally shaped like a hollow box. The bottom surface is square and slanted slightly downwards towards the back. The bottom also contains drain holes 24 situated next to the back wall. The walls of the basin 22 extend vertically from the bottom. The back wall is the highest wall and contains a mounting location for the nozzle 30. The side walls of the basin 22 slope downward towards the front wall. A side wall contains the mounting location for the control mechanism 26, but the control mechanism could be located on any outer surface of the basin 22. The front wall is the shortest wall and curved slightly concave up. In alternative embodiments the basin 22 could have mounting locations for any number of additional sensory engagement elements. The basin 22 is made from injection molded plastic, but could also be made out of fiberglass or machined aluminum. The purpose is to collect water from the nozzle 30 and drain water back into the removable water reservoir 16. The basin 22 is affixed directly above the base 10 using interlocking fasteners, or other such fastening means.

The control mechanism 26 is rectangular and contains the on/off toggle switch 28 for the pump 32. In alternative embodiments, the control mechanism 26 could contain on/off switches for any number of additional sensory engagement elements. The control mechanism 26 is located on the top of one of the side walls of the basin 22, but could be located on any outer surface of the basin 22. The control mechanism 26 is affixed to the basin 22 by screws, or other such fastening means. The purpose is to make the on/off toggle switch 28 for the pump 32 easily accessible by the user and not in the water.

The nozzle 30 is a rectangular box with a domed top and an opening on one face of the box. The open face is oriented towards the basin 22. The nozzle 30 is affixed on the back wall of the basin 22 using screws, or other such fastening means. The bottom side of the nozzle 30 has an opening to allow insertion of a tube. The nozzle 30 is made from injection molded plastic, but could also be made out of fiberglass or machined aluminum. The purpose is to allow water to flow from the removable water reservoir 16 into the basin 22.

The pump 32 is generally cylindrical and housed inside the base 10. The pump 32 is made out of injection molded plastic, but could be made out of machined aluminum. A check valve 34 connects the pump 32 to the removable water reservoir 16. A hose 36 connects the pump 32 to the nozzle 30. The pump 32 is oriented in such a way as to pull water out of the removable water reservoir 16 through the check valve 34 and then push the water through the hose 36 to the nozzle 30. The pump 32 is affixed to the base 10 by screws, or other such fastening means. The purpose is to circulate water from the removable water reservoir 16 into the nozzle 30. This invention is used to provide a safe environment for special needs and other children to interact with sensory elements such as running water and soothing sounds. The removable water reservoir 16 houses the water. The pump 32 pulls water from the removable water reservoir 16 and sends it through the nozzle 30. The water coming out of the nozzle 30 will land in the basin 22, pool up temporarily, and drain through the drain holes 24 back into the removable water reservoir 16. The control mechanism 26 allows the user to turn the pump 32 on and off. The control mechanism 26 also allows the user to turn on and off any additional sensory elements that are present in the specific embodiment. The removable water reservoir 16 can be removed from the base 10 to allow easy fill up and disposal of the water. The water system as a whole is small enough to be portable and placed on top of a table, if desired. The previously described versions of the present invention have many advantages, including a circulatory water flow, a removable water reservoir, and safe use. However, the invention does not require that all the advantageous features and all the advantages need to be incorporated into every embodiment of the invention. By having a circulatory flow water system, a child can play with water in a way that does not run up a family's water bill. By having a removable water reservoir, each individual user of the system can have their own fresh water instead of using the water of the previous user. By having no access to electrical components, the autistic child is in no danger of shock or harm from coming into contact with electricity while playing in the water. By having no temperature control, the user is not in danger of burning one's skin while using the system.

1. A circulatory flow water sensory system comprising:
(a) a base;
(b) a water reservoir which is removable and housed inside said base;
(c) a basin affixed on top of said base with a drain hole to allow any water in the basin to drain out;
(d) a control mechanism mounted on said basin;
(e) a nozzle affixed on the back wall of said basin; and
(f) a pump mounted inside said base and connected to said nozzle and said water reservoir and electrically connected to said control mechanism.

2. A circulatory flow water sensory system as recited in claim 1 further comprising a locking mechanism such that said water reservoir can be locked into said base.

3. A circulatory flow water sensory system as recited in claim 1 further comprising an LED display mounted on said basin and electrically connected to said control mechanism.

4. A circulatory flow water sensory system as recited in claim 1 further comprising a basin with a drain hole positioned in such a way as to allow the water to drain directly into said water reservoir.
